(19) United States
(12) Patent Application Publication
(10) Pub. No.: US 2016/0192859 A1
(43) Pub. Date: Jul. 7, 2016

(54) MAGNETIC RESONANCE IMAGING APPARATUS AND TEMPERATURE INFORMATION MEASUREMENT METHOD

(71) Applicant: HITACHI MEDICAL CORPORATION, Tokyo (JP)

(72) Inventors: Toru SHIRAI, Tokyo (JP); Suguru YOKOSAWA, Tokyo (JP); Hisaaki OCHI, Tokyo (JP); Yoshitaka BITO, Tokyo (JP)

(21) Appl. No.: 14/916,051

(22) PCT Filed: Aug. 5, 2014
(86) PCT No.: PCT/JP2014/070564

§ 371 Date: Mar. 3, 2016

(30) Foreign Application Priority Data
Sep. 9, 2013 (JP) 2013-186592

(51) Int. Cl. A61B 5/055; A61B 5/01; A61B 5/00

(52) U.S. Cl. CPC A61B 5/055; A61B 5/0042; A61B 5/01; A61B 5/7257

(57) ABSTRACT

A technique for improving accuracy of temperature measurement in a living body using MRS/MRSI is provided. A cerebrospinal fluid suppression sequence that does not affect nuclear magnetic resonance signals of metabolite, but suppresses nuclear magnetic resonance signals of cerebrospinal fluid is executed in advance of execution of a signal measurement sequence for measuring nuclear magnetic resonance signals of water and a desired metabolite. There are thereby obtained spectra of water and the metabolite obtained from the nuclear magnetic resonance signals of water and the metabolite in which nuclear magnetic resonance signals of cerebrospinal fluid is suppressed. The obtained spectral peaks are fitted to a model function to obtain resonant frequencies of water and the metabolite, and the difference thereof is used to calculate temperature.

### TECHNICAL FIELD

The present invention relates to a magnetic resonance imaging technique, especially techniques of magnetic resonance spectroscopy (MRS) and magnetic resonance spectroscopic imaging (MRSI), in which types of molecules, components etc. in living bodies are examined by utilizing difference of resonant frequencies of substances.

### BACKGROUND ART

Magnetic resonance imaging apparatuses are apparatuses that irradiate a radio frequency magnetic field of a specific frequency on a measurement object placed in a static magnetic field to induce a magnetic resonance phenomenon, and thereby obtain physical or chemical information of the measurement object. Magnetic resonance imaging (MRI) widely used today is a method of imaging difference of hydrogen nucleus density, relaxation time, or the like varying depending on types of biological tissues by mainly using the nuclear magnetic resonance phenomenon of hydrogen nucleus in water molecule. This technique enables imaging of difference in tissues, and is achieving notable effects in diagnoses of diseases.

On the other hand, MRS and MRSI are methods of separating nuclear magnetic resonance signals for every molecule on the basis of the difference in the resonance frequency (chemical shift) caused by differences of chemical bonds in molecules (metabolites), and measuring differences of density, relaxation time, or the like of respective molecular species. MRS is a method of observing molecular species in a certain selected spatial region, and MRSI is a method of imaging each kind of molecular species. Examples of the target atomic nucleus include those of ¹H (proton), ³¹P, ¹³C, ¹⁹F, and so forth.

The major metabolites contained in human bodies and detectable by the proton MRS or proton MRSI (henceforth simply referred to as MRS/MRSI), which uses proton as the target atom species, include choline, creatine, N-acetylaspartate (NAA), lactate, and so forth. It is hoped to perform noninvasive determination of progression rate, early diagnoses, and malignancy diagnoses of metabolic disorders such as cancers on the basis of amounts of those metabolites.

There is a method for measuring temperature in a living body by using this MRS/MRSI (for example, refer to Non-patent document 1). It is known that the resonance frequency of water shifts depending on temperature, and amount of the shift is represented with a temperature coefficient of −0.01 ppm/° C. It is also known that, on the other hand, the resonant frequencies of metabolites such as NAA do not change in the temperature range of the biological environment. By using these characteristics, temperature in a living body is measured from difference of resonant frequencies of water and metabolite.

### PRIOR ART REFERENCE

### Non-Patent Document

Non-patent document 1: Cady E. B. et al., The Estimation of Local Brain Temperature by in Vivo 1H Magnetic Resonance Spectroscopy, Magnetic Resonance in Medicine, 1995, vol. 33, pages 862-867

### SUMMARY OF THE INVENTION

### Object to be Achieved by the Invention

According to the method of Non-patent document 1, temperature in a living body is calculated in accordance with the conversion equation described in the reference using difference of resonant frequencies of water and metabolite. The resonant frequencies of water and the metabolite are obtained by separately or simultaneously measuring spectra of water and the metabolite by MRS/MRSI, and fitting the obtained spectral peaks to a model function including the resonant frequencies of water and the metabolite as parameters.

Since the resonant frequencies of the substances are obtained by fitting to a model function, if the shape of the measured spectral peak is distorted, fitting accuracy is degraded, and accuracy of the calculated temperature is also degraded. For example, when the imaging object is brain, the major factors that cause distortion of the spectrum of water are contamination of cerebrospinal fluid in a voxel of the measurement object (region of interest). This is caused because T₁ and T₂ of the signals of the cerebrospinal fluid are longer than T₁ and T₂ of the signals of the brain parenchyma, and signals of a substance showing different T₁ and T₂ are mixed.

Furthermore, it is also considered that, in the case of MRSI, because of the few measurement matrices, the point spread function is degraded, and signals of the surrounding cerebrospinal fluid are mixed.

Therefore, in order to improve accuracy of the temperature measurement using MRS/MRSI, it is necessary to improve the shape of the spectral peak of water. Further, in order to improve the shape of the spectral peak of water, it is necessary to sufficiently suppress signals of cerebrospinal fluid.

The present invention was accomplished in view of the aforementioned technical situation, and an object of the present invention is to provide a technique for improving accuracy of the measurement of temperature in a living body using MRS/MRSI.

### Means for Achieving the Object

According to the present invention, a cerebrospinal fluid suppression sequence that does not affect nuclear magnetic resonance signals of metabolites, but suppresses nuclear magnetic resonance signals of cerebrospinal fluid is executed in advance of execution of a signal measurement sequence for measuring nuclear magnetic resonance signals of water and a desired metabolite. There are thereby obtained spectra of water and the metabolite obtained from the nuclear magnetic resonance signals of water and the metabolite in which nuclear magnetic resonance signals of cerebrospinal fluid are suppressed. The obtained spectral peaks are fitted to a model function to obtain resonant frequencies of water and the metabolite, and the difference thereof is used to calculate temperature.

### Effect of the Invention

According to the present invention, in the temperature measurement using MRS/MRSI, accuracy of the measurement of temperature in a living body is improved.

### BRIEF DESCRIPTION OF THE DRAWINGS

FIG. 1, (a) is an exterior view of an MRI apparatus of the horizontal magnetic field type as the MRI apparatus of the first embodiment. FIG. 1, (b) is an exterior view of an MRI apparatus of the vertical magnetic field type as the MRI apparatus of the first embodiment. FIG. 1, (c) is an exterior view of an MRI apparatus comprising a leaned tunnel-shaped magnet as the MRI apparatus of the first embodiment.

FIG. 2 is a functional configuration diagram of the MRI apparatus of the first embodiment.

FIG. 3, (a) is an explanatory drawing for explaining the voxel position in MRS measurement performed without contamination of cerebrospinal fluid, FIG. 3, (b) is an explanatory drawing for explaining the voxel position in MRS measurement performed with slight contamination of cerebrospinal fluid, FIG. 3, (c) is an explanatory drawing for explaining the voxel position in MRS measurement performed with contamination of cerebrospinal fluid, FIG. 3, (d) is a graph showing the shape of water spectrum peak at the voxel position of FIG. 3, (a), FIG. 3, (e) is a graph showing the shape of water spectrum peak at the voxel position of FIG. 3, (b), and FIG. 3, (f) is a graph showing the shape of water spectrum peak at the voxel position of FIG. 3, (c).

FIG. 4 is a functional block diagram of the computer of the first embodiment.

FIG. 5 is a flowchart of the temperature measurement processing of the first embodiment.

FIG. 6 is an explanatory diagram for explaining an example of the cerebrospinal fluid suppression sequence of the first embodiment.

FIG. 7 is an explanatory diagram for explaining an example of the signal measurement sequence of the first embodiment.

FIGS. 8, (a) to (c) are drawings for explaining a region excited by the signal measurement sequence of the first embodiment.

FIG. 9 is a flowchart of the temperature information calculation processing of the first embodiment.

FIG. 10 is a graph showing the results of the computer simulation of the signal measurement of the first embodiment.

FIG. 11 is an explanatory diagram for explaining another example of the cerebrospinal fluid suppression sequence of the first embodiment.

FIG. 12 is a diagram showing an example of the cerebrospinal fluid suppression sequence of the second embodiment.

FIG. 13 is a functional block diagram of a computer used in a modification of the second embodiment.

FIG. 14 is a flowchart of the flip angle setting processing of the second embodiment.

FIGS. 15, (a) and (b) are graphs showing the results of the computer simulation of signal measurement according to the second embodiment.

### MODES FOR CARRYING OUT THE INVENTION

### First Embodiment

Hereafter, embodiments of the present invention will be explained. In all the appended drawings for explaining the embodiments referred to below, the same numerical symbols are assigned to elements having the same function, and repetitive explanations thereof will be omitted.

### <Exterior View of MRI Apparatus>

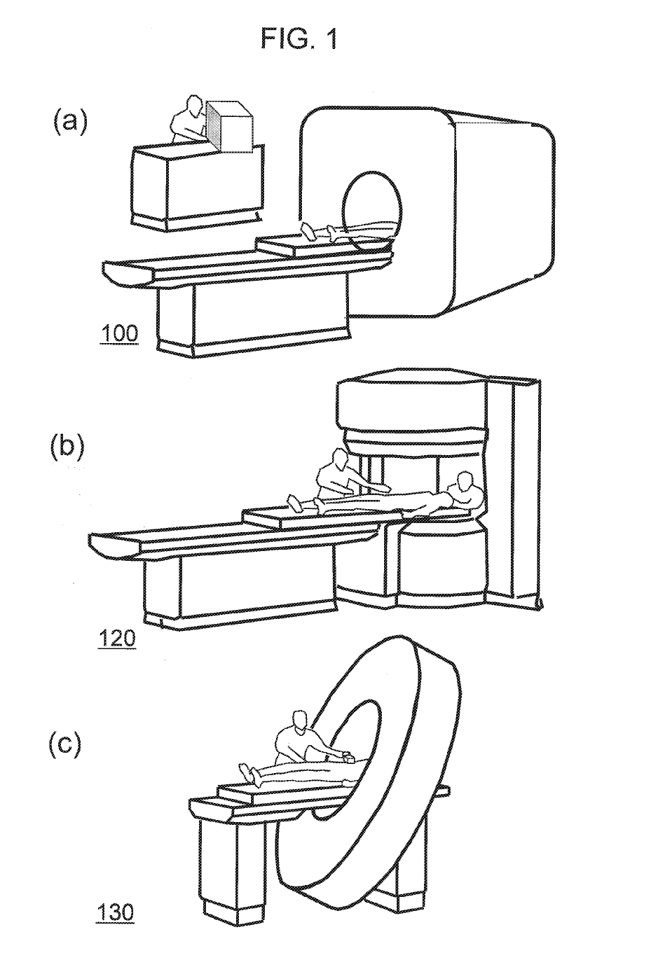

First, the magnetic resonance imaging apparatus (MRI apparatus) of this embodiment will be explained. FIGS. 1, (a) to (c) are exterior views of the MRI apparatuses of this embodiment. FIG. 1, (a) shows an MRI apparatus 100 of the horizontal magnetic field type using a tunnel-shaped magnet that generates a static magnetic field with a solenoid coil. FIG. 1, (b) shows a hamburger type (open type) MRI apparatus 120 of the vertical magnetic field type, which comprises separate upper and lower magnets for enhancing spaciousness. FIG. 1, (c) shows an MRI apparatus 130 using a tunnel-shaped magnet similar to that shown in FIG. 1, (a), in which the magnet has a shorter depth and is leaned in order to enhance spaciousness.

In this embodiment, any of the MRI apparatuses of these exterior views can be used. In addition, these are mere examples, and the MRI apparatus of this embodiment is not limited to these configurations. In this embodiment, various kinds of known MRI apparatuses can be used irrespective of shapes and types of the apparatuses. In the following descriptions, the present invention will be explained by exemplifying the MRI apparatus 100 as a typical example, so long as it is unnecessary to specify an MRI apparatus of a particular type.

### <Functional Configuration of MRI Apparatus>

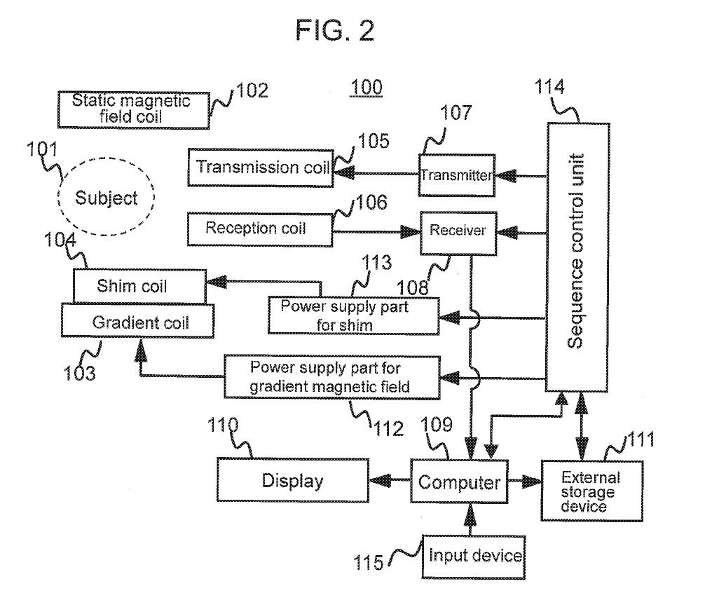

FIG. 2 is a functional configuration diagram of the MRI apparatus 100 of this embodiment. As shown in the diagram, the MRI apparatus 100 of this embodiment comprises a static magnetic field coil 102 that generates a static magnetic field in a space in which a subject 101 is placed, a gradient coil 103 that generates a gradient magnetic field for each of x-, y-, and z-axis directions, a shim coil 104 that adjusts static magnetic field distribution, a radio frequency coil 105 for measurement that irradiates a radio frequency magnetic field to a measurement region of the subject 101 (henceforth simply referred to as transmission coil), a radio frequency coil 106 for reception that receives nuclear magnetic resonance signals generated by the subject 101 (henceforth simply referred to as reception coil), a transmitter 107, a receiver 108, a computer 109, a power supply part 112 for gradient magnetic field, a power supply part 113 for shim, and a sequence control unit 114.

The static magnetic field coil 102 is chosen from those of various forms depending on the structure of the MRI apparatus such as those of the MRI apparatuses 100, 120, and 130 shown in FIGS. 1 (a), (b), and (c), respectively. The gradient coil 103 and the shim coil 104 are driven by the power supply part 112 for gradient magnetic field, and the power supply part 113 for shim, respectively. This embodiment will be explained with an example employing separate transmission coil 105 and reception coil 106, but the apparatus may be constituted with one coil that function as both the transmission coil 105 and the reception coil 106. The radio frequency magnetic field irradiated by the transmission coil 105 is generated by the transmitter 107. Nuclear magnetic resonance signals detected by the reception coil 106 are sent to the computer 109 via the receiver 108.

The sequence control unit 114 controls operations of the power supply part 112 for gradient magnetic field, which is a power supply for driving the gradient coil 103, the power supply part 113 for shim, which is a power supply for driving the shim coil 104, the transmitter 107, and the receiver 108 to control timing of applications of the gradient magnetic field and the radio frequency magnetic field, and reception of nuclear magnetic resonance signals. The time chart for the control is called pulse sequence, and it is determined beforehand according to the measurement, and stored in a storage device or the like of the computer 109 described later.

The computer 109 controls operations of the whole MRI apparatus 100, performs various operation processings with the received nuclear magnetic resonance signals, and generates image information, spectrum information, and temperature information. The functions realized by the computer 109 will be explained later. The computer 109 is an information processor comprising CPU, memory, storage device, and so forth, and a display 110, an external storage device 111, an input device 115, and so forth are connected to the computer 109.

The display 110 is an interface that displays results obtained by the operation processings to an operator. The input device 115 is an interface for the operator to input conditions, parameters, and so forth required for the operation processings performed in this embodiment. The external storage device 111 stores data used for various kinds of operation processings performed by the computer 109, data obtained by the operation processings, inputted conditions, parameters, and so forth, together with the storage device of the computer 109.

### <Distortion of Spectral Peak Caused by Cerebrospinal Fluid>

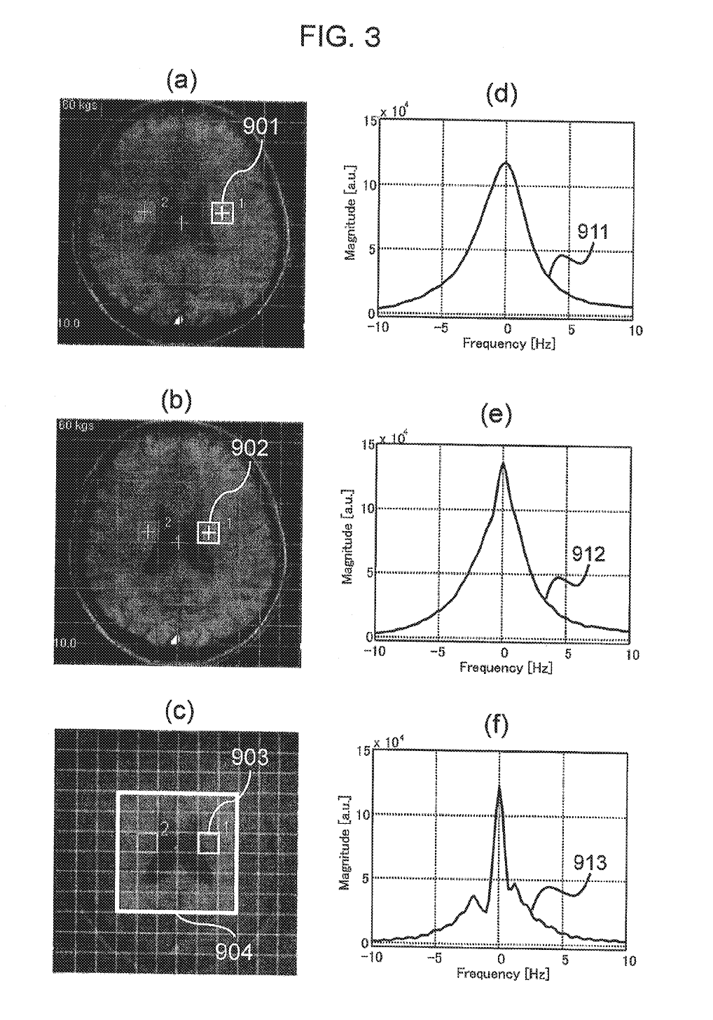

In this embodiment, in the temperature measurement using MRS/MRSI, signals of cerebrospinal fluid is suppressed to improve accuracy of measurement of temperature in a living body. Before explanation of the functions of the computer 109 of this embodiment for realizing the above, influence of cerebrospinal fluid contaminated in the measurement voxel is explained with reference to FIGS. 3, (a) to (f).

FIG. 3, (a) shows a voxel position 901 in MRS measurement performed without contamination of cerebrospinal fluid, FIG. 3, (b) shows an voxel position 902 in MRS measurement performed with slight contamination of cerebrospinal fluid, and FIG. 3, (c) shows a voxel position in MRSI measurement performed with contamination of cerebrospinal fluid in a region of interest 904 (the same voxel position as the voxel position 902 shown in FIG. 3, (b)). FIGS. 3, (d), (e), and (f) shows the spectral peaks 911, 912, and 913 of water at the voxel positions 901, 902, and 903, respectively.

As shown in FIG. 3, (e), it can be seen that, because of contamination of cerebrospinal fluid, the spectral peak 912 has a shape different from that of the spectral peak 911 shown in FIG. 3, (d) obtained without contamination of cerebrospinal fluid. This is because the signals of cerebrospinal fluid showing T₁ and T₂ longer than those of the brain parenchyma are mixed.

Further, as shown in FIG. 3, (f), it can be seen that, when MRSI measurement is performed, the spectral peak 913 has a shape further different from the peak shape obtained by performing MRS measurement, and the distortion is more significant. It is considered that this is because, in addition to the contamination of signals of cerebrospinal fluid showing T₁ and T₂ longer than T₁ and T₂ of the brain parenchyma, the few measurement matrices of MRSI degrade the point spread function, and signals of surrounding cerebrospinal fluid are mixed.

As described above, when temperature information is calculated by using MRS/MRSI, resonant frequencies of substances are obtained by fitting spectral peaks to a model function. Therefore, if the shape of the measured spectral peak is distorted, the fitting accuracy is degraded, and accuracy of temperature to be calculated is also degraded. One of the major factors that cause distortion of the spectrum of water is contamination of cerebrospinal fluid into a voxel for measurement (region of interest).

In this embodiment, in the temperature measurement using MRS/MRSI, signals of cerebrospinal fluid is suppressed to improve accuracy of the measurement of temperature in a living body. For this purpose, a cerebrospinal fluid suppression sequence which suppresses nuclear magnetic resonance signals of cerebrospinal fluid is executed in advance of signal measurement using MRS/MRSI. In this specification, a nuclear magnetic resonance signal of a substance may be henceforth simply referred to as a signal of a substance.

### <Functional Configuration of Computer>

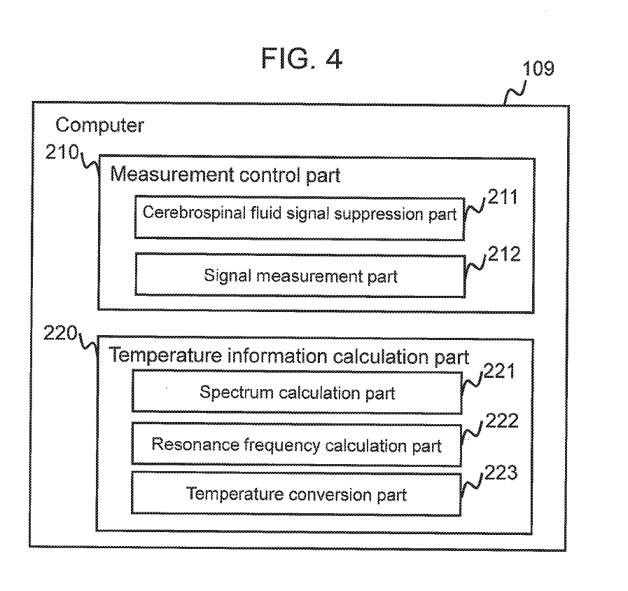

Hereafter, the functions realized by the computer 109 of this embodiment will be explained. FIG. 4 is a functional block diagram of the computer 109 of this embodiment.

As shown in this diagram, the computer 109 of this embodiment comprises a measurement control part 210 that controls the parts of the MRI apparatus 100 so that, after the nuclear magnetic resonance signals of cerebrospinal fluid are suppressed without affecting the nuclear magnetic resonance signals of a metabolite, nuclear magnetic resonance signals of water (water signals) and nuclear magnetic resonance signals of the metabolite (metabolite signals) other than those of cerebrospinal fluid are measured, and a temperature information calculation part 220 that calculates temperature information of a subject from the nuclear magnetic resonance signals obtained by the measurement control part 210.

The measurement control part 210 comprises a cerebrospinal fluid signal suppression part 211 that executes a cerebrospinal fluid suppression sequence for suppressing nuclear magnetic resonance signals of cerebrospinal fluid without affecting the nuclear magnetic resonance signals of the metabolite, and a signal measurement part 212 that executes a signal measurement sequence for measuring nuclear magnetic resonance signals of water and a desired metabolite immediately after the execution of the cerebrospinal fluid suppression sequence.

The temperature information calculation part 220 comprises a spectrum calculation part 221 that converts the nuclear magnetic resonance signals of water and the desired metabolite obtained by the signal measurement part 212 with the signal measurement sequence into spectra, a resonance frequency calculation part 222 that obtains resonant frequencies of water and the metabolite from the converted spectra, respectively, and a temperature conversion part 223 that converts difference of both the resonant frequencies into temperature to obtain temperature information of a subject.

The various kinds of functions realized by the computer 109 are realized by CPU by loading programs stored in the storage device into a memory, and executing them. One or more of the various kinds of functions realized by the computer 109 may be realized by an information processor that is independent from the MRI apparatus 100, and can transmit and receive data to and from the MRI apparatus 100. Further, all or a part of the functions may be realized by hardware such as ASIC (application specific integrated circuit) and FPGA (field-programmable gate array).

The pulse sequences of the cerebrospinal fluid suppression sequence to be executed by the cerebrospinal fluid signal suppression part 211 and the signal measurement sequence to be executed by the signal measurement part 212 are stored beforehand in the storage device of the computer 109 or the external storage device 111. Further, the imaging parameters for defining them are stored beforehand in these storage devices, or set by a user and stored in these storage devices. Various kinds of other data used for processings for the functions and various kinds of data generated during the processings are stored in the storage device or the external storage device 111.

### <Flow of Temperature Information Measurement Processing>

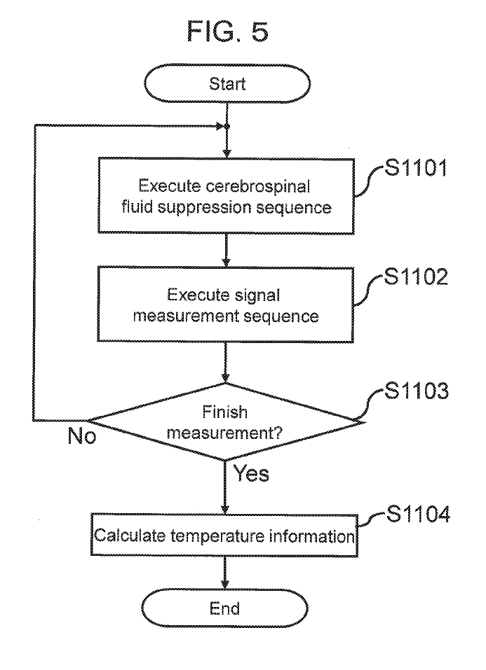

Hereafter, the flow of the whole temperature measurement processing of this embodiment performed by the aforementioned parts using MRS/MRSI will be briefly explained. FIG. 5 shows the process flow of the temperature measurement processing of this embodiment.

According to this embodiment, signals of cerebrospinal fluid are suppressed, and then water signals and metabolite signals other than those of the cerebrospinal fluid are measured. Therefore, the cerebrospinal fluid signal suppression part 211 first executes the cerebrospinal fluid suppression sequence defined beforehand (step S1101). In this operation, according to the cerebrospinal fluid suppression sequence, the sequence control unit 114 is controlled to suppress the nuclear magnetization of the cerebrospinal fluid.

Immediately after the execution of the cerebrospinal fluid control sequence, the signal measurement part 212 executes the signal measurement sequence (step S1102). In this process, the sequence control unit 114 is controlled according to the signal measurement sequence defined beforehand to obtain water signals and signals of the desired metabolite in a state that signals of cerebrospinal fluid are suppressed. Hereafter, this embodiment will be explained for an example where the desired metabolite is NAA.

The measurement control part 210 repeats the execution of the cerebrospinal fluid suppression sequence, and the following execution of the signal, measurement sequence until a predetermined condition for ending the measurement such as number of times of addition or number of steps of phase encoding is satisfied (step S1103).

Then, the temperature information calculation part 220 calculates temperature information by using the nuclear magnetic resonance signals of water and the nuclear magnetic resonance signals of NAA, in which nuclear magnetic resonance signals of cerebrospinal fluid are suppressed (step S1104).

### Example of Cerebrospinal Fluid Suppression Sequence

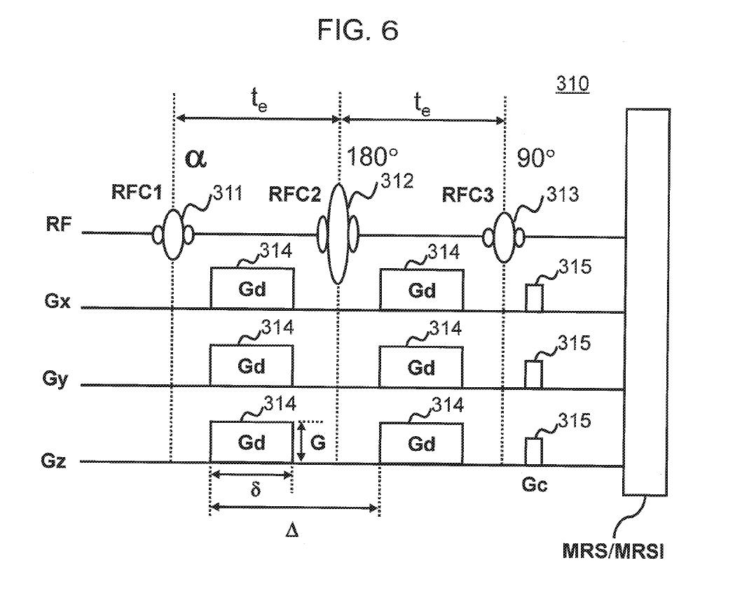

Hereafter, an example of the cerebrospinal fluid suppression sequence to be executed by the cerebrospinal fluid signal suppression part 211 will be explained. FIG. 6 shows an example of the cerebrospinal fluid suppression sequence 310 of this embodiment. In FIG. 6, RF represents application time of a radio frequency magnetic field pulse. Gx, Gy, and Gz represent application times of the gradient magnetic field pulses for the x-, y-, and z-axis directions, respectively. The same shall apply to the following descriptions in this specification.

As shown in FIG. 6, the cerebrospinal fluid suppression sequence 310 comprises a narrow band frequency-selective excitation pulse (RFC1) 311 for selectively exciting only nuclear magnetization of water, a narrow band frequency-selective inversion pulse (RFC2) 312 for selectively reversing only the transverse magnetization of water, a frequency-selective flip back pulse (RFC3) 313 for converting the transverse magnetization of water into longitudinal magnetization, diffusion-weighted gradient magnetic field pulses (Gd) 314 for attenuating the nuclear magnetic resonance signals of cerebrospinal fluid applied before and after the frequency-selective inversion pulse (RFC2) 312, and spoiler gradient magnetic field pulses (Gc) 315 for spoiling the transverse magnetization components of water remaining after the application of the frequency-selective flip back pulse (RFC3) 313.

The irradiation interval between the frequency-selective excitation pulse (RFC1) 311 and the frequency-selective inversion pulse (RFC2) 312, and the irradiation interval between the frequency-selective inversion pulse (RFC2) 312 and the frequency-selective flip back pulse (RFC3) 313 are represented as tₑ. The time tₑ is determined beforehand so that a desired value of the diffusion factor b value, which will be explained later, can be realized within a range allowed by the hardware.

The cerebrospinal fluid signal suppression part 211 first irradiates the narrow band frequency-selective excitation pulses (RFC1) 311 for selectively exciting only nuclear magnetization of water. The flip angle α of this frequency-selective excitation pulse (RFC1) 311 is set to be a value defined beforehand. The value of the flip angle to be set is a value of 90° or smaller, and should be such a value that the intensity of water signal should not saturate even at a reception gain that provides the maximum SNR of the signal intensity of the metabolite. All the water including that contained in cerebrospinal fluid is thereby excited, and transverse magnetization is generated. However, the metabolite is not influenced.

Then, after the time tₑ, the narrow band frequency-selective inversion pulse (RFC2) 312 for selectively reversing only the transverse magnetization of water is irradiated to reverse the transverse magnetization of all the water including that of cerebrospinal fluid. The flip angle of this frequency-selective inversion pulse (RFC2) 312 is set to be 180°.

Then, further after the time tₑ, the narrow band frequency-selective flip back pulse (RFC3) 313 for selectively flipping back only the transverse magnetization of water is irradiated. The time of the application of this pulse is the time when spin echo signals are generated by the frequency-selective excitation pulse (RFC1) 311 and the frequency-selective inversion pulse (RFC2) 312. The transverse magnetization of all the water including that of cerebrospinal fluid is converted into longitudinal magnetization. The flip angle of this frequency-selective flip back pulse (RFC3) 313 is set to be 90°.

After the frequency-selective flip back pulse (RFC3) 313 is irradiated, the spoiler gradient magnetic field (Gc) 315 for spoiling the remaining transverse magnetization components of water is applied.

Further, one set of diffusion-weighted gradient magnetic field pulses (Gd) 314 are applied before and after the frequency-selective inversion pulse (RFC2) 312 for reversing the transverse magnetization of water. The signals of cerebrospinal fluid are thereby attenuated and suppressed.

The principle of the attenuation of the signals of cerebrospinal fluid with the diffusion-weighted gradient magnetic field pulse (Gd) 314 will be explained below.

First, transverse magnetization of stationary water without molecular diffusion is supposed. In the case of transverse magnetization of stationary water without molecular diffusion, the dephasing amount provided by the diffusion-weighted gradient magnetic field pulse (Gd) 314 applied before the frequency-selective inversion pulse (RFC2) 312, and the rephasing amount provided by the diffusion-weighted gradient magnetic field pulse (Gd) 314 applied after the frequency-selective inversion pulse (RFC2) 312 can be balanced. Therefore, all the transverse magnetization once dephased is rephrased, and attenuation of signal amount is not caused for the macroscopic magnetization, which means the total magnetization.

On the other hand, if there is molecular diffusion, the position of the transverse magnetization once dephased already shifts at the time of rephasing. Therefore, in such transverse magnetization, the dephasing amount and rephrasing amount are different, and the dephased magnetization is not fully rephased. Accordingly, the signal amount is attenuated for the macroscopic magnetization.

The attenuation amount is represented by the following equation (1) described with a coefficient representing magnitude of molecular diffusion, and the b value of the diffusion-weighted gradient magnetic field pulse (Gd) 314.

[Equation 1]

S(b)=S₀exp(−bD) (1)

In the equation, S(b) is signal intensity when the b value is b, S0 is signal intensity when the b value is 0, and D is a diffusion coefficient.

The b value [s/mm²] is a diffusion factor, which is a parameter concerning application intensity and application time of the MPG pulse. The b value is a value determined by application intensity G, application time δ, and application interval Δ of the diffusion-weighted gradient magnetic field pulse (Gd) 314, and is calculated in accordance with the following equation (2).

[Equation 2]

b=∫₀^{τ}γ²|∫₀^{t}G(τ)dτ|²dt (2)

In the above equation, τ is time [s] from the irradiation of the frequency-selective excitation pulses (RFC1) 311 to the irradiation of the frequency-selective flip back pulse (RFC3) 313, γ is nuclear magnetogyric ratio [Hz/μT], and G(τ) is gradient magnetic field application intensity [μT/mm] at the time τ. When the diffusion-weighted gradient magnetic field pulse (Gd) 314 is applied as two pulses, in particular, the b value is calculated in accordance with the following equation (3).

[Equation 3]

b=γ²G²δ²(Δ−δ/3) (3)

In the above equation, G is application intensity of diffusion gradient magnetic field [μT/mm], δ is application time [s] of one diffusion-weighted gradient magnetic field pulse (Gd) 314, and Δ is application interval [s] of two diffusion-weighted gradient magnetic field pulses (Gd) 314.

Water contained in the brain parenchyma, typically white matter, gray matter, etc., generally shows restricted diffusion, in which diffusion area is restricted by cell walls. On the other hand, water in cerebrospinal fluid is approximate liquid not restricted by cells, and therefore it substantially shows free diffusion. Therefore, the diffusion coefficient D of cerebrospinal fluid is several times larger than the diffusion coefficient D of water in the brain parenchyma. For this reason, by applying the diffusion-weighted gradient magnetic field pulse (Gd) 314, water signals of cerebrospinal fluid can be reduced relative to water signals of the brain parenchyma.

The b value that defines the magnitude of the diffusion-weighted gradient magnetic field pulse (Gd) 314 to be applied is set to be a value within a range of the value realizable by the hardware on the basis of a value that provides the desired suppression effect estimated from simulation results etc.

In the example shown in FIG. 6, the diffusion-weighted gradient magnetic field pulses (Gd) 314 and the spoiler gradient magnetic field pulses (Gc) 315 are applied for all the x-, y-, and z-axis directions. However, this embodiment is not limited to such a configuration. The diffusion-weighted gradient magnetic field pulse (Gd) 314 and the spoiler gradient magnetic field (Gc) 315 may be applied for at least one axis direction among the x-, y-, and z-axis directions. Further, the flip angle α of the frequency-selective excitation pulses (RFC1) 311 may be set to be an arbitrary value other than 90°.

### <Signal Measurement Sequence>

Hereafter, an example of the signal measurement sequence to be executed by the signal measurement part 212 will be explained. According to this embodiment, for example, either an MRS sequence or an MRSI sequence is used as the signal measurement sequence. A pulse sequence for region-selective type magnetic resonance spectroscopic imaging for imaging metabolite (henceforth referred to as MRSI sequence) is explained below as an example.

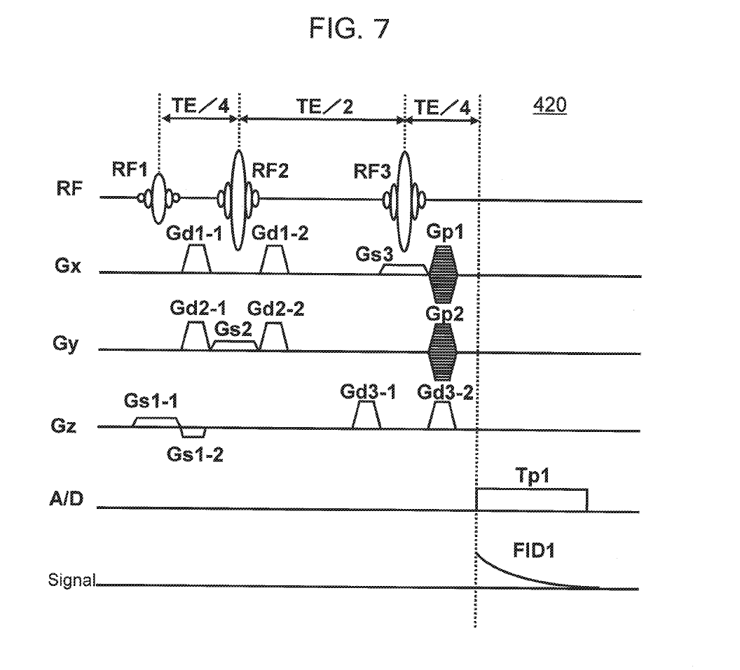

FIG. 7 shows an example of the MRSI pulse sequence (signal measurement sequence) 420. In FIG. 7, A/D represents a signal measurement period. The same shall apply to the following descriptions in this specification.

The MRSI pulse sequence 420 shown in FIG. 7 is the same as known MRSI pulse sequences, and selectively excites a predetermined region of interest (voxel) by using one excitation pulse (RF1), which is a radio frequency magnetic field pulse, and two inversion pulses (RF2) and (RF3) to obtain a FID (free induction decay) signal FID1 from this region of interest (voxel).

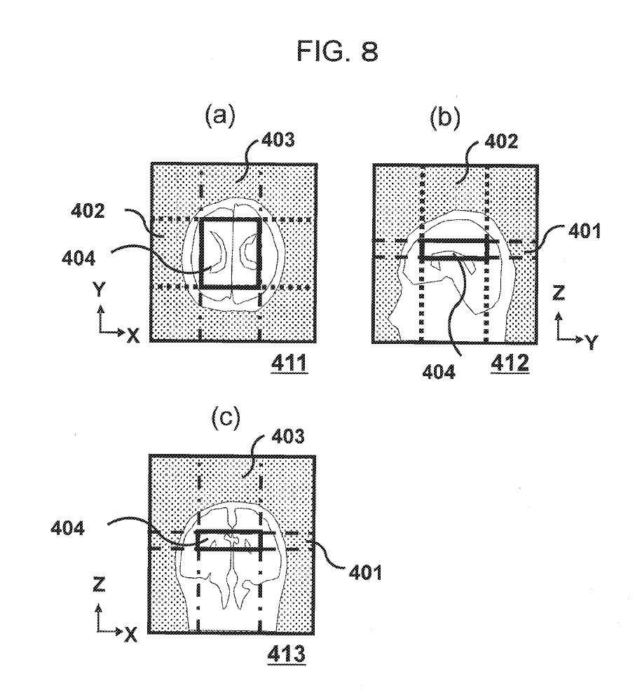

Images of the region excited according to this MRSI pulse sequence 420 is shown in FIGS. 8, (a) to (c). The images shown in FIGS. 8, (a) to (c) are scout images for positioning obtained by a measurement performed in advance of signal measurement, and are trans image 411 (FIG. 8, (a)), sagittal image 412 (FIG. 8, (b)), and coronal image 413 (FIG. 8, (c)), respectively. Hereafter, the relations between the operations of the parts and the region to be excited will be explained with reference to FIGS. 7 and 8.

First, the excitation pulse (RF1) and the gradient magnetic field pulses for the z-axis direction (Gs1-1) and (Gs1-2) are applied to excite a section perpendicular to the z-axis (henceforth referred to simply as section of the z-direction) 401. After the time TE/4 (TE is echo time), the inversion pulse (RF2) and the gradient magnetic field pulse (Gs2) for the y-axis direction are applied. As a result, only the phase of the nuclear magnetization in the crossing region of the section 401 of the z-direction, and the section perpendicular to the y-axis (section of the y-direction) 402 is rephased (returned).

Then, after the time TE/2 from the application of the inversion pulse (RF2), the inversion pulse (RF3) and the gradient magnetic field pulse (Gs3) for the x-axis direction are applied. Only the phase of the nuclear magnetization in the region of interest 404 where the section 401 of the z-direction, the section 402 of the y-direction, and a section perpendicular to the x-axis (section of x-direction) 403 are crossing is thereby rephrased, and a free induction decay signal (FID1) is generated from the region. This free induction decay signal (FID1) is measured.

The gradient magnetic field pulses (Gd1-1), (Gd2-1) (Gd3-1), (Gd1-2), (Gd2-2), and (Gd3-2) for those directions are gradient magnetic field pulses for rephasing the phase of the nuclear magnetization excited by the excitation pulse (RF1), and dephasing the phase of the nuclear magnetization excited by the inversion pulse (RF2) and the inversion pulse (RF3). Further, after the inversion pulse (RF3), a phase encoding gradient magnetic field pulse (Gp1) and a phase encoding gradient magnetic field pulse (Gp2) are applied. By the above operation, a nuclear magnetic resonance signal of the region of interest 404 is obtained.

### <Calculation of Temperature Information>

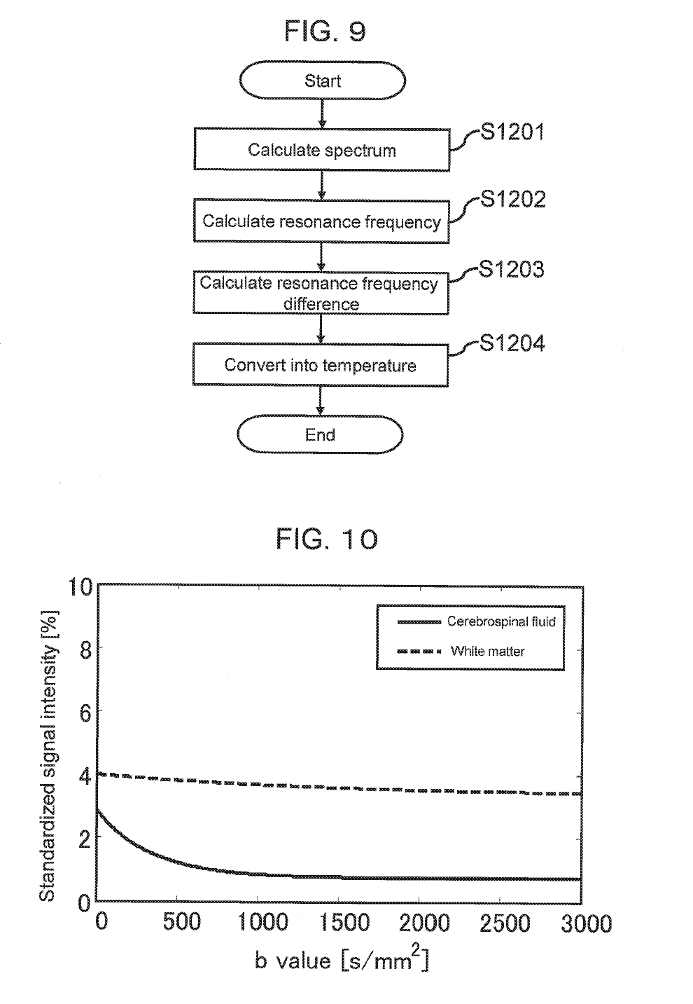

Hereafter, temperature information calculation processing performed by the temperature information calculation part 220 will be explained. FIG. 9 shows a flowchart for explaining the flow of the temperature information calculation processing of this embodiment. According to this embodiment, the spectral peaks of water and NAA are fitted to a model function to calculate resonant frequencies of them, and the difference thereof is converted into temperature.

First, the spectrum calculation part 221 performs the Fourier transform of the nuclear magnetic resonance signal of water and the nuclear magnetic resonance signal of NAA, which are obtained with the signal measurement sequence, in the direction of time, to calculate the spectrum of water, and the spectrum of NAA, respectively (step S1201).

Then, the resonance frequency calculation part 222 fits the spectral peak of water and the spectral peak of NAA to a model function to calculate resonant frequencies of them (step S1202).

As the model functions, for example, the Lorenz function represented by the following equation (4) is used.

$$\begin{matrix}[ Equation 4 ] & \\ {L}_{i} ( v ) = \frac{{a}_{i}^{2} {I}_{i}}{{a}_{i}^{2} + 4 {( v - {v}_{i} )}^{2}} cos {φ}_{i} + \frac{2 {a}_{i} {I}_{i} ( v - {v}_{i} )}{{a}_{i}^{2} + 4 {( v - {v}_{i} )}^{2}} sin {φ}_{i} + c & ( 4 )\end{matrix}$$

In the equation, ν is frequency, Lᵢ is signal intensity, νᵢ is resonance frequency of an objective substance, aᵢ is half width of spectral peak, Iᵢ is height of spectral peak, φᵢ is phase, and c is an absolute term.

The measured spectral peak of water and spectral peak of NAA are each fitted to the model function represented by the equation (4) to obtain a resonance frequency ν_{W} of water and resonance frequency ν_{NAA} of NAA, respectively, as resonant frequencies νᵢ as the parameters.

Then, the temperature conversion part 223 calculates difference Δν (difference of resonant frequencies) of the resonance frequency of water and the resonance frequency of NAA (step S1203).

Further, the temperature conversion part 223 converts the calculated difference of the resonant frequencies into temperature by using a temperature conversion equation for converting frequency difference into temperature (step S1204). As the temperature conversion equation, for example, the following equation (5) is used.

T=A×Δν+B (5)

In the equation, T is temperature, A is a coefficient having a dimension of temperature/frequency, and B is an absolute term. As A and B in the equation (5), known values mentioned in references or experimentally obtained values are used.

### <Computer Simulation>

Hereafter, it is demonstrated by computer simulation that signals of cerebrospinal fluid can be suppressed by executing the cerebrospinal fluid suppression sequence 310 of this embodiment immediately before the signal measurement sequence 420. The results of simulation performed by executing the cerebrospinal fluid suppression sequence 310, and then the signal measurement sequence 420 to obtain signals of cerebrospinal fluid, and simulation performed in the same manner to obtain signals of white matter are shown in FIG. 10.

In the simulation, T₁, T₂, and the diffusion coefficient D of a cerebrospinal fluid model were 4000 [ms], 2000 [ms], and 3.0×10⁻³ [mm²/s], respectively, and T₁, T₂, and the diffusion coefficient D of a white matter model were 556 [ms], 79 [ms], and 0.7×10⁻³ [mm²/s], respectively. Further, in the cerebrospinal fluid suppression sequence 310, the flip angle of the frequency-selective excitation pulses (RFC1) 311 was 5°, and the time tₑ was 80 [ms]. In the signal measurement sequence 420, the repetition time TR was 1500 [ms], and the echo time TE was 35 [ms].

FIG. 10 is a graph obtained by plotting the signal intensity of the cerebrospinal fluid, and the signal intensity of the white matter against the varying b value of the diffusion-weighted gradient magnetic field pulse (Gd) 314. The signal intensity was standardized on the basis of the magnitudes (proton density) of the nuclear magnetization of the cerebrospinal fluid model and the white matter model, which were taken as 100%.

As shown in FIG. 10, it can be seen that the signal of the cerebrospinal fluid is smaller than the signal of the white matter. It can also be seen that the intensity of the signal of the white matter does not substantially change even when the b value changes. In contrast, it can be seen that the signal intensity of cerebrospinal fluid more reduces when the b value is made larger, and when the b value becomes about 1000 [s/mm²] or larger, the signal intensity becomes substantially constant.

The above results demonstrated that the signals of cerebrospinal fluid can be suppressed with respect to the signals of brain parenchyma such as white matter by the method of this embodiment. It was demonstrated that, in a range of the b value smaller than the certain value, the suppressing effect is more improved by making the b value larger. Therefore, according to this embodiment, since the signal measurement is performed in a state that the signals of cerebrospinal fluid are suppressed, distortion of the obtained spectral peak of water decreases, and since temperature is calculated on the basis of the peak including less distortion, accuracy of the measurement of temperature in a living body is improved.

As explained above, the MRI apparatus 100 of this embodiment comprises the cerebrospinal fluid signal suppression part 211 that executes the cerebrospinal fluid suppression sequence 310 for suppressing the nuclear magnetic resonance signals of cerebrospinal fluid, the signal measurement part 212 that executes the signal measurement sequence 420 for measuring nuclear magnetic resonance signals of water and a desired metabolite immediately after the cerebrospinal fluid suppression sequence 310, and the temperature information calculation part 220 that calculates temperature information of a subject from the nuclear magnetic resonance signals of water and the desired metabolite obtained with the signal measurement sequence 420.

Further, the cerebrospinal fluid suppression sequence 310 comprises the frequency-selective excitation pulses 311 that selectively excites only nuclear magnetization of water, the frequency-selective inversion pulse 312 that selectively reverses only the transverse magnetization of water, the frequency-selective flip back pulse 313 that converts the transverse magnetization of water to longitudinal magnetization, and the diffusion-weighted gradient magnetic field pulses 314 for attenuating the nuclear magnetic resonance signal of cerebrospinal fluid applied before and after the frequency-selective inversion pulse 312.

As described above, in this embodiment, the cerebrospinal fluid suppression sequence that does not affect the signals of metabolite and suppresses signals of cerebrospinal fluid is executed before performing the signal measurement. According to this embodiment, by utilizing the fact that the diffusion coefficient D of cerebrospinal fluid is several times as large as the diffusion coefficient D of water in the brain parenchyma, suppression of the nuclear magnetic resonance signals of cerebrospinal fluid is realized with the frequency-selective pulse that acts only on nuclear magnetization of water, and the diffusion-weighted gradient magnetic field pulse. Further, water signals are measured in a state that signals of cerebrospinal fluid are suppressed.

Distortion of the spectral peak of water caused by signals of cerebrospinal fluid can be thereby reduced. Thus, a spectral peak including less distortion can be obtained, and accuracy of the measurement of temperature in a living body, which is calculated by using the spectral peak, can be improved.

### Other Example of Cerebrospinal Fluid Suppression Sequence

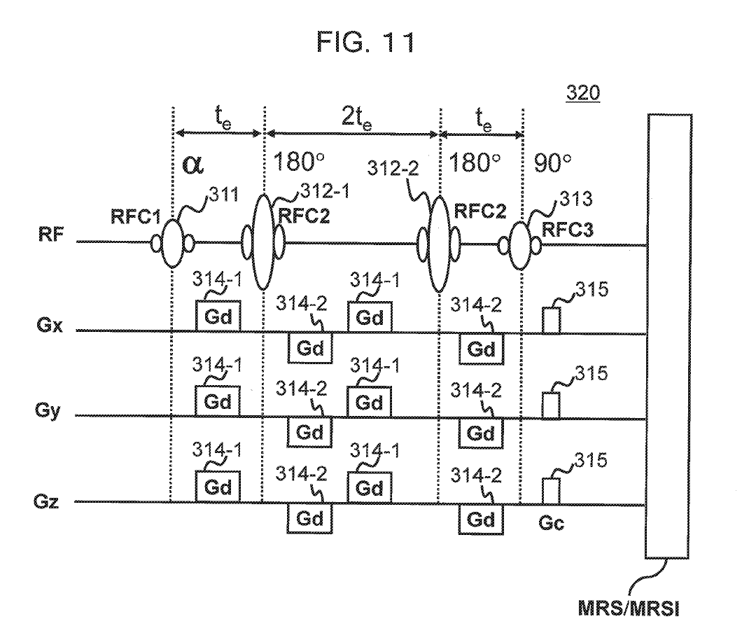

The cerebrospinal fluid suppression sequence to be executed by the cerebrospinal fluid signal suppression part 211 is not limited to the aforementioned cerebrospinal fluid suppression sequence 310. Another example thereof will be explained below. FIG. 11 shows an example of the cerebrospinal fluid suppression sequence 320 of this embodiment.

As shown in FIG. 11, this cerebrospinal fluid suppression sequence 320 comprises the narrow band frequency-selective excitation pulse (RFC1) 311 for selectively exciting only nuclear magnetization of water, a plurality of the narrow band frequency-selective inversion pulses (RFC2) 312 for selectively reversing only the transverse magnetization of water, the frequency-selective flip back pulse (RFC3) 313 for converting the transverse magnetization of water into longitudinal magnetization, the diffusion-weighted gradient magnetic field pulse (Gd) 314 for attenuating nuclear magnetic resonance signals of cerebrospinal fluid, which is applied before and after each frequency-selective inversion pulse (RFC2) 312, and the spoiler gradient magnetic field pulse (Gc) 315 for spoiling transverse magnetization components of water remaining after irradiation of the frequency-selective flip back pulse (RFC3) 313.

The plurality of frequency-selective inversion pulses (RFC2) 312 are successively irradiated between the frequency-selective excitation pulses (RFC1) 311 and the frequency-selective flip back pulse (RFC3) 313. Further, one set of the diffusion-weighted gradient magnetic field pulses (Gd) 314 applied before and after one frequency-selective inversion pulse (RFC2) 312 are applied with alternately changed polarity for every frequency-selective inversion pulse (RFC2) 312. FIG. 11 exemplifies a case where the frequency-selective inversion pulse (RFC2) 312 is irradiated twice.

The irradiation interval between the frequency-selective excitation pulses (RFC1) 311 and the frequency-selective inversion pulse (RFC2) 312, and the irradiation interval between the frequency-selective inversion pulse (RFC2) 312 and the frequency-selective flip back pulse (RFC3) 313 are set to be tₑ. The irradiation interval of the frequency-selective inversion pulses (RFC2) 312 is set to be 2tₑ.

The cerebrospinal fluid signal suppression part 211 first irradiates the narrow band frequency-selective excitation pulse (RFC1) 311 for selectively exciting only nuclear magnetization of water. The flip angle α of this frequency-selective excitation pulse (RFC1) 311 is set to be a predetermined value α, as in the cerebrospinal fluid suppression sequence 310. All the water including that of the cerebrospinal fluid is thereby excited, and transverse magnetization is generated.

Then, after the time tₑ, the frequency-selective inversion pulse (RFC2) 312 is irradiated to reverse the transverse magnetization of all the water including that of cerebrospinal fluid. Further, after the time 2tₑ, the frequency-selective inversion pulse (RFC2) 312 is irradiated again to reverse the transverse magnetization of all the water including that of cerebrospinal fluid. Also in this sequence, the flip angle of the frequency-selective inversion pulse (RFC2) 312 is set to be 180°. In FIG. 11, the frequency-selective inversion pulse (RFC2) that is irradiated the first time is indicated with 312-1, and the frequency-selective inversion pulse (RFC2) that is irradiated the second time is indicated with 312-2.

Then, after the time tₑ thereafter, the frequency-selective flip back pulse (RFC3) 313 is irradiated to convert the transverse magnetization of all the water including that of cerebrospinal fluid to longitudinal magnetization. The flip angle of this frequency-selective flip back pulse (RFC3) 313 is set to be 90°.

After the irradiation of the frequency-selective flip back pulse (RFC3) 313, the spoiler gradient magnetic field pulse (Gc) 315 is applied.

With the cerebrospinal fluid suppression sequence 320, the diffusion-weighted gradient magnetic field pulses (Gd) 314 are applied before and after each of the two frequency-selective inversion pulses (RFC2) 312. In FIG. 11, the diffusion-weighted gradient magnetic field pulses (Gd) that are applied before and after the frequency-selective inversion pulse (RFC2) 312-1 are indicated with 314-1, and the diffusion-weighted gradient magnetic field pulses (Gd) applied before and after the frequency-selective inversion pulse (RFC2) 312-2 are indicated with 314-2.

The diffusion-weighted gradient magnetic field pulse (Gd) 314-1, and the diffusion-weighted gradient magnetic field pulse (Gd) 314-2 are applied with inverted polarities. FIG. 11 shows an example where the diffusion-weighted gradient magnetic field pulse (Gd) 314-1 is applied with positive polarity, and the diffusion-weighted gradient magnetic field pulse (Gd) 314-2 is applied with negative polarity. As already explained, by applying these diffusion-weighted gradient magnetic field pulses (Gd) 314-1 and 314-2, the signals of cerebrospinal fluid can be suppressed.

The number of times of the irradiation of the frequency-selective inversion pulse (RFC2) 312 is determined so that the total of the b values of the diffusion-weighted gradient magnetic field pulses (Gd) 314 applied by the whole cerebrospinal fluid suppression sequence 320 corresponds to or exceeds the objective b value.

Further, also in this cerebrospinal fluid suppression sequence 320, the diffusion-weighted gradient magnetic field pulse (Gd) 314 may be applied for at least one axis directions among the x-axis, y-axis, and z-axis directions.

Although this cerebrospinal fluid suppression sequence 320 extends the time of the cerebrospinal fluid suppression sequence compared with the aforementioned cerebrospinal fluid suppression sequence 310, it enables a plurality of times of application of the set of the frequency-selective inversion pulse (RFC2) 312 and the diffusion-weighted gradient magnetic field pulse (Gd) 314. For example, even when the desired b value cannot be attained with one time of irradiation of the diffusion-weighted gradient magnetic field pulse (Gd) 314 due to restrictions imposed by the apparatus or the like, the desired b value can be attained by repeating the application a plurality of times. Therefore, signals of cerebrospinal fluid can be suppressed irrespective of restrictions imposed by the apparatus.

### Second Embodiment

Hereafter, the second embodiment of the present invention will be explained. According to the first embodiment, signals of cerebrospinal fluid are suppressed by applying the frequency-selective pulse that acts only on nuclear magnetization of water, and the diffusion-weighted gradient magnetic field pulse as pre-pulses. In contrast, in this embodiment, a plurality of frequency-selective CHESS pulses are irradiated as pre-pulses to suppress signals of cerebrospinal fluid.

The MRI apparatus 100 of this embodiment has basically the same configuration as that of the first embodiment. The functional configuration realized by the computer 109 is also the same. However, the pre-pulses applied for suppressing signals of cerebrospinal fluid differ as described above. Therefore, the cerebrospinal fluid suppression sequence is different. Hereafter, explanation of this embodiment will be made with being focused on the configuration different from that of the first embodiment.

According to this embodiment, the frequency-selective excitation pulse (CHESS pulse) for selectively exciting only nuclear magnetization of water is irradiated at least two times or more as the cerebrospinal fluid suppression sequence. Further, the spoiler gradient magnetic field pulses of different intensities are applied after the pulses to spoil the transverse magnetization components of water signals (phase-diffused). Signals of cerebrospinal fluid of longer T₁ and T₂ are thereby suppressed. The flip angle of the above frequency-selective excitation pulse is set to be a predetermined value β.

### Example of Cerebrospinal Fluid Suppression Sequence

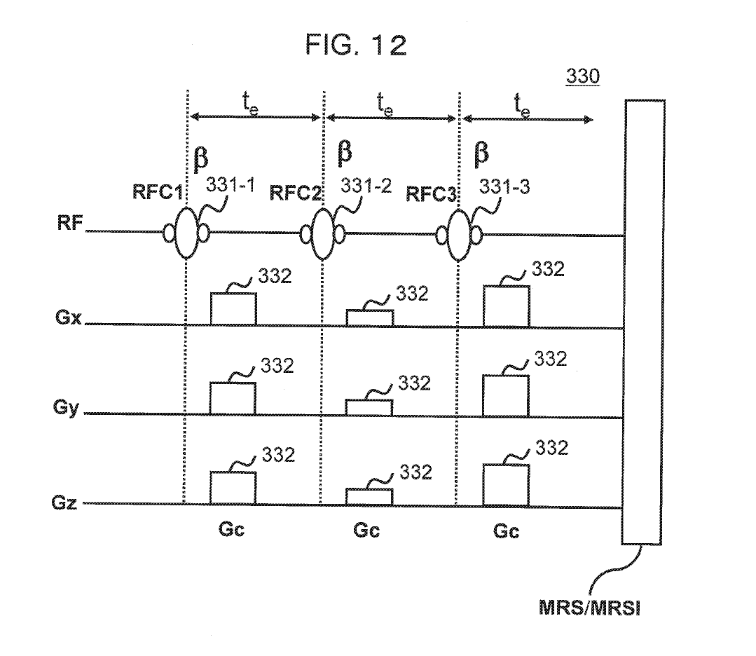

An example of the cerebrospinal fluid suppression sequence to be executed by the cerebrospinal fluid signal suppression part 211 of this embodiment will be explained. FIG. 12 shows an example of the cerebrospinal fluid suppression sequence 330 of this embodiment.

As shown in FIG. 12, the cerebrospinal fluid suppression sequence 330 of this embodiment includes a plurality of frequency-selective excitation pulses (RFC) 331 for selectively exciting nuclear magnetization of water, and spoiler gradient magnetic field pulses (Gc) 332 for spoiling remaining transverse magnetization components of water to be applied upon every application of the above frequency-selective excitation pulses.

The number of times of the irradiation (number of pulses) of the frequency-selective excitation pulses (RFC) 331 is represented as N (N is an integer of 1 or larger). When a plurality of the frequency-selective excitation pulses (RFC) 331 are distinguished, the frequency-selective excitation pulse irradiated n-th time (n is an integer not smaller than 1 and not larger than N) is represented as (RFCn) 331-n. FIG. 12 shows a case where N is 3.

The flip angle β of each frequency-selective excitation pulse (RFC) 331 is set to be a predetermined value. The predetermined value is, for example, 90°.

The irradiation interval of the frequency-selective excitation pulses (RFC) 331 is set to be tₑ. The irradiation interval tₑ is set to be the possible shortest interval (shortest time) in consideration of the irradiation time of the frequency-selective excitation pulses (RFC) 331, and the application time of the spoiler gradient magnetic field Gc. By setting the irradiation interval tₑ of frequency-selective excitation pulses to be short, signals of cerebrospinal fluid of longer T₁ and T₂ compared with those of the brain parenchyma can be suppressed.

The number of times N of the irradiation of the frequency-selective excitation pulse (RFC) 331 is set to be the maximum number of the pulses that can be irradiated with the aforementioned irradiation interval within a time in which the cerebrospinal fluid suppression sequence 330 can be executed, which is determined by the repetition time TR and the time required for the signal measurement sequence.

The number of times n of the irradiation of the frequency-selective excitation pulse (RFC) 331 may be determined in further consideration of specific absorption rate (SAR). That is, it is determined to be the smaller number among the aforementioned maximum number and the maximum number determined in consideration of restriction imposed by SAR.

Intensities of the spoiler gradient magnetic field pulses (Gc) 332 are each set to be such an intensity that gradient echo, spin echo, or stimulated echo is not generated by the irradiation of a plurality of frequency-selective excitation pulses (RFC) 331. Further, for example, the intensity of each spoiler gradient magnetic field pulse 332 is set to be an intensity that is not an integral multiple of the intensity of the first spoiler gradient magnetic field pulse 332.

Therefore, in this embodiment, the cerebrospinal fluid signal suppression part 211 irradiates the narrow band frequency-selective excitation pulses (RFC) 331 for selectively exciting only nuclear magnetization of water N times with the time interval tₑ. Further, after the irradiation of each frequency-selective excitation pulses (RFC) 331, each spoiler gradient magnetic field pulse (Gc) 332 for spoiling the remaining transverse magnetization components of water is applied.

### <Temperature Measurement Processing>

The flow of the temperature measurement processing performed by the parts of the apparatus of this embodiment is the same as that of the first embodiment except that the aforementioned cerebrospinal fluid suppression sequence 330 is used as the cerebrospinal fluid suppression sequence.

### <Setting of Flip Angle>

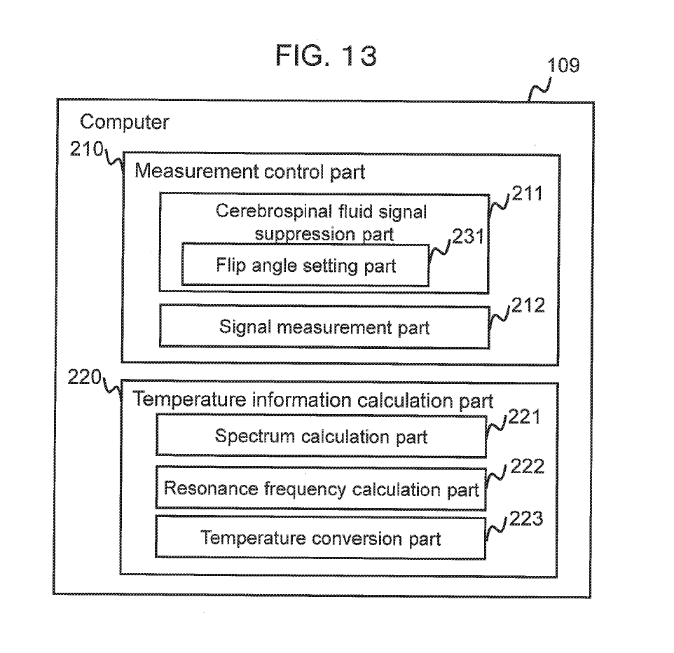

In addition, the cerebrospinal fluid signal suppression part 211 may comprises a flip angle setting part 231 as shown in FIG. 13, and this flip angle setting part 231 may set the flip angle β of the frequency-selective excitation pulses (RFC) 331 thorough the procedure describes below.

In this explanation, in order to determine the flip angle to be used at the time of the actual measurement (main measurement), the flip angle setting part 231 executes the same sequences as the cerebrospinal fluid suppression sequence 330 and the signal measurement sequence 420 with changing an initially set flip angle by a predetermined degree, and sets a value corresponding to a feature point of an approximated curve of the nuclear magnetic resonance signal group of water as the flip angle of the frequency-selective excitation pulse to be used in the main measurement. When the number of times N of the irradiation of the frequency-selective excitation pulse is an even number, a point corresponding to the minimum value is used as the feature point, and when the number N is an odd number, a point corresponding to the inflexion point is used as the feature point.

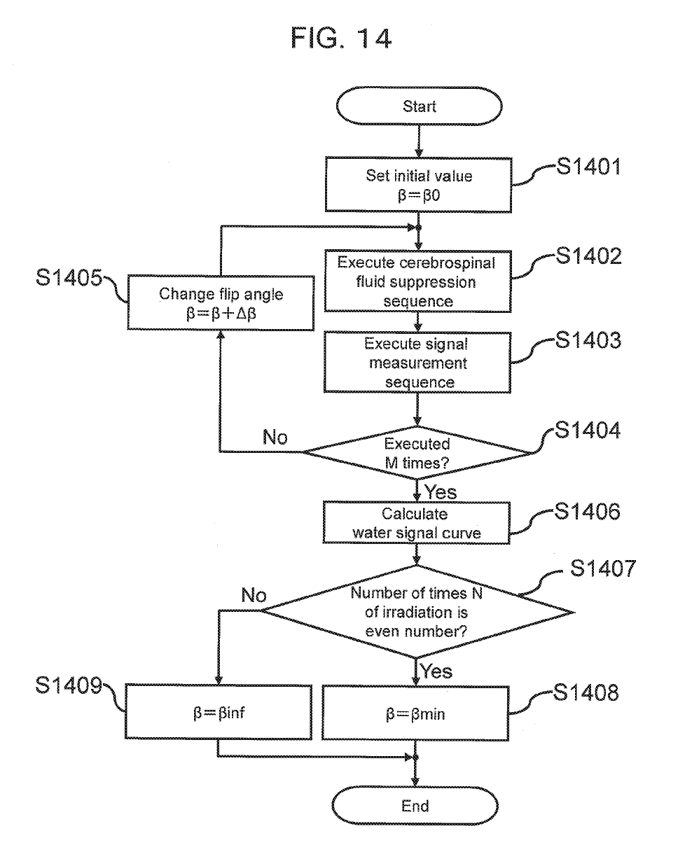

The procedure for setting the flip angle 3 of the frequency-selective excitation pulses (RFC) 331 to be executed by the flip angle setting part 231 is explained with reference to the process flow shown in FIG. 14.

First, the flip angle setting part 231 sets the flip angle β of the frequency-selective excitation pulses (RFC) 331 to be an arbitrary value (initial value β0) (step S1401). Then, the same sequence as the cerebrospinal fluid suppression sequence 330 is executed (step S1402), and then the same sequence as the signal measurement sequence 420 is successively executed (step S1403) to measure the nuclear magnetic resonance signals of water.

The flip angle setting part 231 repeats the aforementioned steps S1401 to S1403 M times as a predetermined repetition number of times (step S1404), with continuously changing the flip angle β of the frequency-selective excitation pulses (RFC) 331 (step S1405). For this operation, changing amount Δβ of the flip angle is defined beforehand. M is an integer of 3 or larger.

The flip angle setting part 231 calculates a curve of water signal varying with variation of the flip angle β of the frequency-selective excitation pulses (RFC) 331 by using M of water signals acquired by M times of the measurement (step S1406). A continuous water signal curve is obtained by fitting M of the discrete water signal values to an N-th order function of the order of the same number as the number of times N of the irradiation.

Then, the flip angle setting part 231 judges whether the number of times n of the irradiation of the frequency-selective excitation pulses (RFC) 331 is an even number or not (step S1407).

When it is an even number, a flip angle βmin that provides the minimum value in a narrow range around the flip angle of 90° is calculated, and used as the flip angle β of the frequency-selective excitation pulses (RFC) 331 (step S1408).

When it is an odd number, a flip angle βinf that provides an inflexion point in a narrow range around the flip angle of 90° is calculated, and used as the flip angle β of the frequency-selective excitation pulses (RFC) 331 (step S1409).

A stable flip angle β can be adjusted by the above procedure even when spatial non-uniformity of the flip angle differs for every subject 101.

### <Simulation Results>

Hereafter, it is demonstrated that, by executing the cerebrospinal fluid suppression sequence 330 of this embodiment immediately before the signal measurement sequence 420, signals of cerebrospinal fluid can be suppressed, and a larger number of times of the irradiation of the frequency-selective excitation pulses (RFC) 331 in the cerebrospinal fluid suppression sequence 330 results in less influence of the setting error of the flip angle β of the frequency-selective excitation pulses (RFC) 331.

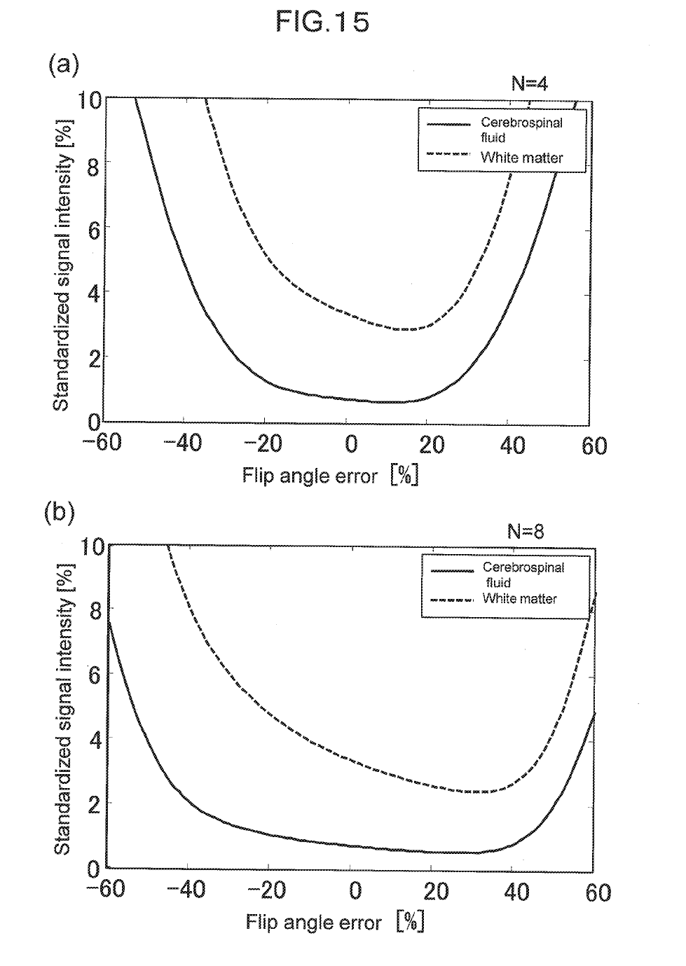

The results of the simulation of obtaining signals of cerebrospinal fluid, and simulation of obtaining signals of white matter, which were performed by executing the cerebrospinal fluid suppression sequence 330, and then the signal measurement sequence 420, are shown in FIGS. 15, (a) and (b), respectively.

In the simulations, T₁ and T₂ of a cerebrospinal fluid model were 4000 [ms], and 2000 [ms], respectively, and T₁ and T₂ of a white matter model were 556 [ms], and 79 [ms], respectively. Further, in the cerebrospinal fluid suppression sequence 310, the irradiation interval of the frequency-selective excitation pulses (RFC) 331 tₑ was 30 [ms], and in the signal measurement sequence 420, the repetition time TR was 1500 [ms], and the echo time TE was 35 [ms].

FIG. 15, (a) shows a graph in which signal intensities of the cerebrospinal fluid and white matter are plotted against error of the flip angle β for the number of times N of the irradiation of 4. FIG. 15, (b) shows a graph in which signal intensities of the cerebrospinal fluid and white matter are plotted against error of the flip angle β for the number of times N of the irradiation of 8. The signal intensities are standardized on the basis of the magnitudes of the nuclear magnetization of the cerebrospinal fluid model and white matter model (proton density), which are taken as 100%.

As shown in FIGS. 15, (a) and (b), it can be seen that the signals of cerebrospinal fluid is smaller than those of white matter. It can also be seen that, when the irradiation number N was increased from 4 to 8, the flip angle error range providing high cerebrospinal fluid signal suppression effect becomes larger.

The aforementioned results demonstrated that signals of cerebrospinal fluid can be suppressed relative to signals of brain parenchyma such as white matter by the method of this embodiment. That is, according to this embodiment, the signal measurement is performed in a state that the signals of cerebrospinal fluid are suppressed, and therefore distortion of the obtained spectral peak of water decreases. Further, since temperature is calculated from the peak including less distortion, accuracy of the measurement of temperature in a living body is improved.

As explained above, the MRI apparatus 100 of this embodiment comprises the cerebrospinal fluid signal suppression part 211 that executes the cerebrospinal fluid suppression sequence 330 for suppressing nuclear magnetic resonance signals of cerebrospinal fluid, the signal measurement part 212 that executes the signal measurement sequence 420 for measuring nuclear magnetic resonance signals of water and a desired metabolite immediately after the cerebrospinal fluid suppression sequence 330, and the temperature information calculation part 220 that calculates temperature information of a subject from the nuclear magnetic resonance signals of water and the desired metabolite obtained with the signal measurement sequence 420.

Further, the cerebrospinal fluid suppression sequence 330 includes a plurality of frequency-selective excitation pulses 331 for selectively exciting only nuclear magnetization of water, and the spoiler gradient magnetic field pulses 332 that are applied for every application of the frequency-selective excitation pulses 331 and spoil the remaining transverse magnetization components of water.

According to this embodiment, like the first embodiment, distortion of the spectral peak of water caused by cerebrospinal fluid signals can be reduced, and accuracy of the measurement of temperature in a living body, for which calculation is performed by using the spectral peak, can be improved.

Furthermore, according to this embodiment, even if there is spatial non-uniformity of the flip angle of the frequency-selective excitation pulses (RFC) 331, cerebrospinal fluid signals can be more suppressed compared with the first embodiment by increasing the number of the frequency-selective excitation pulses (RFC) 331.

### DESCRIPTION OF NUMERICAL NOTATIONS

100 . . . MRI apparatus, 101 . . . subject, 102 . . . static magnetic field coil, 103 . . . gradient coil, 104 . . . shim coil, 105 . . . transmission coil, 106 . . . reception coil, 107 . . . transmitter, 108 . . . receiver, 109 . . . computer, 110 . . . display, 111 . . . external storage device, 112 . . . power supply part for gradient magnetic field, 113 . . . power supply part for shim, 114 . . . sequence control unit, 115 . . . input device, 120 . . . MRI apparatus, 130 . . . MRI apparatus, 210 . . . measurement control part, 211 . . . cerebrospinal fluid signal suppression part, 212 . . . signal measurement part, 220 . . . temperature information calculation part, 221 . . . spectrum calculation part, 222 . . . resonance frequency calculation part, 223 . . . temperature conversion part, 231 . . . flip angle setting part, 310 . . . cerebrospinal fluid suppression sequence, 320 . . . cerebrospinal fluid suppression sequence, 330 . . . cerebrospinal fluid suppression sequence, 401 . . . section of z-direction, 402 . . . section of y-direction, 403 . . . section of x-direction, 404 . . . region of interest, 411 . . . trans image, 412 . . . sagittal image, 413 . . . coronal image, 420 . . . signal measurement sequence, 901 . . . voxel position, 902 . . . voxel position, 903 . . . voxel position, 904 . . . region of interest, 911 . . . spectral peak, 912 . . . spectral peak, 913 . . . spectral peak

1. A magnetic resonance imaging apparatus comprising:
a cerebrospinal fluid signal suppression part that executes a cerebrospinal fluid suppression sequence for suppressing nuclear magnetic resonance signals of cerebrospinal fluid,
a signal measurement part that executes a signal measurement sequence for measuring nuclear magnetic resonance signals of water and a desired metabolite immediately after the cerebrospinal fluid suppression sequence, and
a temperature information calculation part that calculates temperature information of a subject from the nuclear magnetic resonance signals of water and the desired metabolite obtained with the signal measurement sequence.

2. The magnetic resonance imaging apparatus according to claim 1, wherein:
the cerebrospinal fluid suppression sequence includes:
a frequency-selective excitation pulse for selectively exciting only nuclear magnetization of water,
a frequency-selective inversion pulse for selectively reversing only transverse magnetization of water,
a frequency-selective flip back pulse for converting the transverse magnetization of water into longitudinal magnetization, and
a diffusion-weighted gradient magnetic field pulse for attenuating nuclear magnetic resonance signals of cerebrospinal fluid, which is applied before and after the frequency-selective inversion pulse.

3. The magnetic resonance imaging apparatus according to claim 2, wherein:
a plurality of the frequency-selective inversion pulses are included, and
the diffusion-weighted gradient magnetic field pulse is applied with alternately inversed polarities for every frequency-selective inversion pulse.

4. The magnetic resonance imaging apparatus according to claim 1, wherein:
the cerebrospinal fluid suppression sequence includes:
a plurality of frequency-selective excitation pulses for selectively exciting only nuclear magnetization of water, and
a spoiler gradient magnetic field pulse for spoiling remaining transverse magnetization components of water, which is applied for every application of the frequency-selective excitation pulse.

5. The magnetic resonance imaging apparatus according to claim 2, wherein:
the diffusion-weighted gradient magnetic field pulse is applied for a direction of at least one axis among x-axis, y-axis, and z-axis.

6. The magnetic resonance imaging apparatus according to claim 2, wherein:
flip angle of the frequency-selective excitation pulse is 90° or smaller,
flip angle of the frequency-selective inversion pulse is 180°, and
flip angle of the frequency-selective flip back pulse is 90°.

7. The magnetic resonance imaging apparatus according to claim 4, wherein:
the flip angle of the frequency-selective excitation pulse is 90°.

8. The magnetic resonance imaging apparatus according to claim 4, wherein:
the cerebrospinal fluid signal suppression part further comprises a flip angle setting part that sets the flip angle of the frequency-selective excitation pulse,
the flip angle setting part executes the same sequences as the cerebrospinal fluid suppression sequence and the signal measurement sequence with changing an initially set flip angle by a predetermined degree, and sets a value corresponding to a feature point of an approximated curve of a nuclear magnetic resonance signal group of water as the flip angle of the frequency-selective excitation pulse to be used in main measurement.

9. The magnetic resonance imaging apparatus according to claim 3, wherein:
number of times of irradiation of the frequency-selective inversion pulse is determined so that total of b values of the diffusion-weighted gradient magnetic field pulses to be applied become a desired value.

10. The magnetic resonance imaging apparatus according to claim 4, wherein:
irradiation interval of the frequency-selective excitation pulses is the minimum time that is determined on the basis of irradiation time of the frequency-selective excitation pulses, and application time of the spoiler gradient magnetic field pulse, and
number of times of irradiation of the frequency-selective excitation pulse is the maximum number of the pulses that can be irradiated within a time in which the cerebrospinal fluid suppression sequence can be executed, and which is determined on the basis of the repetition time.

11. The magnetic resonance imaging apparatus according to claim 4, wherein:
irradiation interval of the frequency-selective excitation pulses is the minimum time that is determined on the basis of irradiation time of the frequency-selective excitation pulses, and application time of the spoiler gradient magnetic field pulse, and
number of times of irradiation of the frequency-selective excitation pulse is not larger than the maximum number of the pulses that can be irradiated within a time in which the cerebrospinal fluid suppression sequence can be executed, and which is determined on the basis of the repetition time, and not larger than a number possible under restrictions imposed by specific absorption rate.

12. The magnetic resonance imaging apparatus according to claim 1, wherein:
the temperature information calculation part comprises:
a spectrum calculation part that converts nuclear magnetic resonance signals of water and a desired metabolite obtained with the signal measurement sequence into spectra,
a resonance frequency calculation part that obtains resonant frequencies of water and the metabolite from the converted spectra, respectively, and
a temperature conversion part that converts difference of the resonance frequency of water and the resonance frequency of the metabolite into temperature to obtain temperature information of the subject.

13. The magnetic resonance imaging apparatus according to claim 1, wherein:
the signal measurement sequence is an MRS (magnetic resonance spectroscopy) sequence or an MRSI (magnetic resonance spectroscopic imaging) sequence.

14. A method for measuring temperature information, which comprises:
executing a cerebrospinal fluid suppression sequence for suppressing nuclear magnetic resonance signals of cerebrospinal fluid, and then executing a signal measurement sequence for measuring nuclear magnetic resonance signals of water and a desired metabolite,
performing the Fourier transform of the nuclear magnetic resonance signals of water and the desired metabolite to calculate spectrum of water and spectrum of the metabolite, respectively,
calculating resonance frequency of water and resonance frequency of the metabolite from the obtained spectrum of water and spectrum of the metabolite, respectively,
calculating difference of the calculated resonance frequency of water and resonance frequency of the metabolite, and
converting the obtained difference of the resonant frequencies to temperature to obtain the temperature information.
